(19) United States
(12) Patent Application Publication
(10) Pub. No.: US 2021/0138260 A1
(43) Pub. Date: May 13, 2021

(54) PHOTOTHERAPY APPRATUS FOR PET

(71) Applicant: BNSoft. Inc., Seoul (KR)

(72) Inventors: Sang Ho PARK, Seongnam-si (KR); Young Joon CHOI, Seoul (KR); Sang Keun PARK, Paju-si (KR)

(73) Assignee: BNSoft. Inc., Seoul (KR)

(21) Appl. No.: 16/683,097

(22) Filed: Nov. 13, 2019

(51) Int. Cl. A61N 5/06

(52) U.S. Cl. CPC A61N 5/0616; A61N 2005/0665; A61N 2005/0644; A61N 2005/0654; A61N 2005/0626; A61N 2005/0663; A61N 2005/0652

(57) ABSTRACT

The inventive concept relates to an apparatus for phototherapy treatment of a pet. The apparatus includes a grippable main body having a pair of heads for respectively emitting light, a plurality of first LED modules respectively disposed in the pair of heads, wherein the plurality of first LED modules irradiate light of a specific wavelength toward a skin and an external auditory meatus of the pet, respectively, at least one second LED module disposed in one of the pair of heads, wherein the at least one second LED module irradiates light of a wavelength different from the wavelength of the light of the plurality of first LED modules toward the external auditory meatus of the pet, and a controller disposed in the main body to control the first LED module and the second LED module to respectively irradiate the light.

### BACKGROUND

Embodiments of the inventive concept described herein relate to a phototherapy treatment apparatus for a pet, and more particularly, relate to a phototherapy treatment apparatus for a pet that may treat ear and skin of the pet using light of a specific wavelength.

In general, dandruff keratin, a mite, a demodex, various other bacteria, and the like are parasitic on a skin and fur of a pet to damage the hair, cause hair loss, and further cause skin diseases frequently.

Therefore, a pet guardian has been used a cleansing agent, a functional shampoo, or the like containing a sterilization component to prevent the diseases of the pet in advance and remove peculiar odor of the pet.

However, the cleansing agent or the functional shampoo has a functional limit because persistence of a sterilization effect and an odor removal effect is very minimal. Therefore, the pet should be washed frequently to prevent the skin disease of the pet. However, when the pet is washed frequently, the fur and skin of the pet may become dry and cause the skin disease.

Further, the pet has been received treatment at an animal hospital, and has been treated with ointment, and oral or injectable medications to treat the skin disease of the pet.

However, due to a nature of the skin disease that requires a long-term steady treatment, excessive use of a chemical synthetic drug containing the sterilization component has a problem of weakening immunity of kidney, liver, and skin tissues of the pet.

In some cases, long-term overuse of the chemical synthetic drug causes fatal damage to the liver or kidney tissues of the pet. As a result, a complication is developed due to the weakening of the immunity resulted from weakening of liver and kidney functions.

In addition, a skin of an external auditory meatus is adversely affected by water introduced into the external auditory meatus of the pet during the washing of the pet. Further, inflammation of the external auditory meatus is caused due to poor air circulation.

Therefore, there is a need for development of a treatment apparatus for a pet that may prevent and manage skin problems of the pet and treat a disease of the external auditory meatus.

### SUMMARY

Embodiments of the inventive concept provide a phototherapy treatment apparatus for a pet that may use light of a specific wavelength to prevent and manage a skin problem and an external auditory meatus disease of the pet, and may be conveniently carried and used regardless of a place.

According to an exemplary embodiment, an apparatus for phototherapy treatment of a pet includes a grippable main body having a pair of heads for respectively emitting light, a plurality of first LED modules respectively disposed in the pair of heads, wherein the plurality of first LED modules irradiate light of a specific wavelength toward a skin and an external auditory meatus of the pet, respectively, at least one second LED module disposed in one of the pair of heads, wherein the at least one second LED module irradiates light of a wavelength different from the wavelength of the light of the plurality of first LED modules toward the external auditory meatus of the pet, and a controller disposed in the main body to control the first LED module and the second LED module to respectively irradiate the light.

Further, the first LED module may emit light having a wavelength of 630 nm, and the second LED module may emit light having a wavelength of 405 nm.

Further, the first LED module and the second LED module may emit light of different colors, respectively.

Further, the apparatus may further include an illumination lamp disposed in at least one of the pair of heads to illuminate the skin or the external auditory meatus of the pet.

Further, the apparatus may further include a light guide detachably coupled to the head to diffuse the light emitted from the head.

Further, the light guide may have a shape of a hollow cone having a constant gradient and having both sides open.

Further, the apparatus may further include a switch disposed on the main body to turn on or off the first LED module and the second LED module.

Further, the apparatus may further include a timer for measuring a time, wherein the controller may control the first LED module and the second LED module irradiating the light to be turned off when a time set by the timer elapses.

Further, the apparatus may further include a battery disposed in the main body to supply power to the first LED module, the second LED module, and the controller.

Further, the apparatus may further include a charging terminal disposed in the main body to charge the battery.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a perspective view of a phototherapy treatment apparatus for a pet according to an embodiment of the inventive concept;

FIG. 2 is an exploded perspective view of FIG. 1;

FIG. 3 is a rear perspective view of FIG. 1;

FIG. 4 is a bottom perspective view of FIG. 1 from which a light guide is removed;

FIG. 5 is a control block diagram of a phototherapy treatment apparatus for a pet according to an embodiment of the inventive concept; and

FIG. 6 is a view illustrating a state of use of a phototherapy treatment apparatus for a pet according to an embodiment of the inventive concept.

### DETAILED DESCRIPTION

Advantages and features of the inventive concept, and methods of achieving them will become apparent with reference to embodiments described below in detail in conjunction with the accompanying drawings. However, the inventive concept is not limited to the embodiments disclosed below, but may be implemented in various forms. The present embodiments are provided to merely complete the disclosure of the inventive concept, and to merely fully inform those skilled in the art of the inventive concept of the scope of the inventive concept. The inventive concept is only defined by the scope of the claims.

The terminology used herein is for the purpose of describing the embodiments only and is not intended to limit the inventive concept. As used herein, the singular forms “a” and “an” are intended to include the plural forms as well, unless the context clearly indicates otherwise. It will be further understood that the terms “comprises”, “comprising”, “includes”, and “including” when used in this specification, specify the presence of the stated features, integers, operations, elements, and/or components, but do not preclude the presence or addition of one or more other features, integers, operations, elements, components, and/or portions thereof. The same reference numbers in different figures denote the same or similar elements, and as such perform similar functionality. As used herein, the term “and/or” includes any and all combinations of one or more of the associated listed items. Although “first”, “second”, etc. are used to describe various components, these components are not limited by these terms. These terms are only used to distinguish one component from another. Therefore, a first component mentioned below may be a second component within a technical spirit of the inventive concept.

Unless otherwise defined, all terms including technical and scientific terms used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Hereinafter, the inventive concept will be described in detail with reference to the accompanying drawings.

FIGS. 1 to 5 illustrate a phototherapy treatment apparatus for a pet according to an embodiment of the inventive concept.

As shown in such drawings, a phototherapy treatment apparatus 1 for a pet according to an embodiment of the inventive concept includes a main body 10, a plurality of first LED modules 20, a single second LED module 30, and a controller 40.

An outer shape of the main body 10 is provided in a grippable streamlined shape such that a pet guardian may hold the main body 10 with a hand thereof and use the treatment apparatus 1.

In addition, the main body 10 has a pair of switches 11 and 13 formed thereon. The switches 11 and 13 are electrically connected to the controller 40 to turn on or off the first LED module 20 and the second LED module 30. For example, the switches include the first switch 11 for turning on or off the first LED module 20 disposed in a first head 15 to be described later and the second switch 13 for simultaneously turning on or off the first LED module 20 and the second LED module 30 disposed in a second head 17 to be described later.

The pair of heads 15 and 17 that emit light are respectively formed at both ends of the main body 10.

Hereinafter, for convenience of description, a head located on a left side of FIG. 1 is referred to as the first head 15, and a head located on a right side of FIG. 1 is referred to as the second head 17.

The first head 15 and the second head 17 in the present embodiment have a rectangular cross-sectional shape, but are not limited thereto. The first head 15 and the second head 17 may have a circular, elliptical, or polygonal cross-sectional shape.

In addition, the second head 17 has a diffusing mirror 19 of a transparent material for diffusing light emitted from the second head 17.

The plurality of first LED modules 20 are arranged in the first head 15 and the second head 17, respectively. The plurality of first LED modules 20 may irradiate light of a specific wavelength toward a skin or an external auditory meatus (110, see FIG. 6) of a pet (100, see FIG. 6).

The first LED module 20 may emit red light of 630 nm wavelength.

Accordingly, as the red light of 630 nm wavelength emitted from each of the first LED modules 20 is irradiated to the skin of the pet 100, the red light of 630 nm wavelength emitted from each of the first LED modules 20 permeates into a dermal layer of skin to induce production of collagen located in the dermal layer of the skin. Thus, a skin cell may be activated, a regenerative power of the skin may be increased, and the skin may be maintained soft.

The second LED module 30 is disposed in the second head 17. The second LED module 30 emits light of a wavelength different from that of the light from the first LED module 20 toward the external auditory meatus 110 of the pet 100.

The second LED module 30 emits light of a wavelength and a color different from those of the light from the first LED module 20. The second LED module 30 may emit blue light having a wavelength of 405 nm.

Accordingly, as the blue light of 405 nm wavelength emitted from each second LED module 30 is irradiated to the external auditory meatus 110 of the pet 100, sterilization and treatment of inflammation occurred in an epidermal layer of the external auditory meatus 110 due to water introduced into the external auditory meatus 110 of the pet 100 is induced, thereby treating a disease of the external auditory meatus 110 and minimizing a skin problem.

As such, as only the first LED module 20 is disposed in the first head 15, and the first LED module 20 and the second LED module 30 are arranged in the second head 17, the phototherapy treatment apparatus 1 for the pet according to an embodiment of the inventive concept may be used by selectively positioning the first head 15 and the second head 17 based on a treatment target part of the pet 100. For example, the phototherapy treatment apparatus 1 for the pet according to an embodiment of the inventive concept may be selectively used for skin treatment of the pet through the first head 15, and for external auditory meatus treatment of the pet through the second head 17.

In this connection, although the second LED module 30 is shown as being disposed only in the second head 17 in the present embodiment, the second LED module 30 may also be disposed in the first head 15.

The controller 40 may be installed in the main body 10 to control the first LED module 20 and the second LED module 30 to irradiate light based on manipulation signals of the first switch 11 and the second switch 13, respectively.

In addition, the phototherapy treatment apparatus 1 for the pet according to an embodiment of the inventive concept may further include an illumination lamp 50 for illuminating light on the skin of the pet 100.

The illumination lamp 50 is electrically connected to the controller 40 and disposed in the first head 15 to be turned on or off by manipulation of the first switch 11. In this connection, the first switch 11 may turn on or off the illumination lamp 50 and the first LED module 20 based on a manipulation sequence. For example, when the first switch 11 is touched once, the controller 40 may transmit a control signal to the illumination lamp 50 such that only the illumination lamp 50 is turned on, when the first switch 11 is touched once again, transmit a control signal to turn on the first LED module 20 after the illumination lamp 50, which was turned on, is turned off, and when the first switch 11 is touched once more, transmit a control signal to turn off the first LED module 20, thereby turning on or off the illumination lamp 50 and the first LED module 20.

As such, the illumination lamp 50 is further included to illuminate the skin epidermis of the pet 100, so that the pet guardian may visually identify an epidermal state of the skin of the pet 100.

In this connection, although the illumination lamp 50 is illustrated as being disposed in the first head 15 in the present embodiment, the inventive concept is not limited thereto. Further, the illumination lamp 50 may be disposed in the second head 17.

In addition, the phototherapy treatment apparatus 1 for the pet according to an embodiment of the inventive concept may further include a light guide 60 detachably coupled to the first head 15.

The light guide 60 has a shape of a hollow cone having a constant gradient and having both sides open, for example, a funnel shape.

A head coupling portion 61, which is connected to the first head 15, is formed at one end of the light guide 60, and a skin contact portion 65, which is in contact with the treatment target part of the pet 100, is formed at the other end thereof.

The head coupling portion 61 has a cross-section having a shape and a size corresponding to the first head 15 such that the head coupling portion 61 is fitted to the first head 15.

As such, as the light guide 60 is fitted to the first head 15, the red light of 630 nm wavelength emitted from the first LED module 20 of the first head 15 may be diffused by the light guide 60 at a predetermined diffusion angle, and irradiated on an irradiation area with uniform illuminance.

In addition, the phototherapy treatment apparatus 1 for the pet according to an embodiment of the inventive concept may further include a timer 70 for measuring time.

The timer 70 is installed in the main body 10, and is operated in association with the controller 40. For example, when a time set by the timer 70 elapses, the controller 40 controls the first LED module 20 and the second LED module 30 that emit light to be turned off.

Further, the phototherapy treatment apparatus 1 for the pet according to an embodiment of the inventive concept may further include a battery 80 and a charging terminal 90.

The battery 80 is disposed in the main body 10, and supplies power to the first LED module 20, the second LED module 30, and the controller 40. The battery 80 also supplies power to the illumination lamp 50.

The charging terminal 90 is disposed in the main body 10 to charge the battery 80. Although a USB port is illustrated in the present embodiment as the charging terminal 90, the inventive concept is not limited thereto. The charging terminal 90 may be formed in various forms.

As such, the battery 80 and the charging terminal 90 are included, so that the phototherapy treatment apparatus 1 for the pet according to an embodiment of the inventive concept may be carried conveniently, and used conveniently regardless of a location.

In this connection, a reference numeral 95, which has not been described, is a charging indicator indicating a state of charge. The charging indicator 95 may blink at regular intervals when charging, and may emit light of a specific color when the charging is completed or when the charging is insufficient.

A process of treating the external auditory meatus 110 of the pet 100 using the phototherapy treatment apparatus 1 for the pet according to an embodiment of the inventive concept with such configuration is described as follows.

As shown in FIG. 6, the second head 17 is placed near the external auditory meatus 110 while the external auditory meatus 110 of the pet 100 is opened.

Further, when the second switch 13 is touched to turn on the first LED module 20 and the second LED module 30 of the second head 17 at the same time, the controller 40 transmits control signals to the first LED module 20 and the second LED module 30 of the second head 17 respectively based on the manipulation signal of the second switch 13, thereby turning on the first LED module 20 and the second LED module 30 of the second head 17. In this connection, when the timer 70 operates and the set time of the timer 70 elapses, the controller 40 transmits control signals to the first LED module 20 and the second LED module 30 respectively to turn off the first LED module 20 and the second LED module 30. Further, when the pet guardian touches the second switch 13 to turn off the first LED module 20 and the second LED module 30 of the second head 17 at the same time before the set time of the timer 70 elapses, the controller 40 transmits the control signals to the first LED module 20 and the second LED module 30 of the second head 17 respectively based on the manipulation signal of the second switch 13, thereby turning off the first LED module 20 and the second LED module 30 of the second head 17.

Further, while the light from the first LED module 20 and the second LED module 30 are irradiated from the second head 17 to the external auditory meatus 110 of the pet 100, the skin problem and disease of the external auditory meatus 110 of the pet 100 may be reduced.

That is, production of collagen positioned in a dermal layer of the external auditory meatus 110 of the pet 100 is induced by the red light of 630 nm wavelength emitted from the first LED module 20, so that the skin cell may be activated, the regenerative power of the skin may be increased, and the skin may be maintained soft. Further, the blue light of 405 nm wavelength emitted from the second LED module 30 induces the sterilization and the treatment of the inflammation occurred in the epidermal layer of the external auditory meatus 110 due to the water introduced into the external auditory meatus 110 of the pet 100, so that the disease of the external auditory meatus 110 may be treated and the skin problem may be minimized.

Further, in a case of managing skin other than the external auditory meatus 110 of the pet 100, while the light guide 60 is mounted to the first head 15, and while the skin contact portion 65 of the light guide 60 is in contact with the treatment target part of the pet 100, the first switch 11 is touched to turn on the first LED module 20 of the first head 15, and then the controller 40 transmits the control signal to the first LED module 20 of the first head 15 based on the manipulation signal of the first switch 11 to turn on the first LED module 20. In this connection, when the timer 70 operates and the set time of the timer 70 elapses, the controller 40 transmits the control signal to the first LED module 20 to turn off the first LED module 20. Further, when the pet guardian touches the first switch 11 to turn off the first LED module 20 of the first head 15 before the set time of the timer 70 elapses, the controller 40 transmits the control signal to the first LED module 20 of the first head 15 based on the manipulation signal of the first switch 11 to turn off the first LED module 20 of the first head 15.

Further, while the light of the first LED module 20 is irradiated from the first head 15 to the skin of the treatment target part of the pet 100, production of collagen positioned in a dermal layer of the treatment target part of the pet 100 is induced by the red light of 630 nm wavelength emitted from the first LED module 20, so that the skin cell may be activated, the regenerative power of the skin may be increased, and the skin may be maintained soft.

In this connection, the light guide 60 may be selectively fitted to the first head 15 as needed. Further, as the light guide 60 is fitted to the first head 15, the red light of 630 nm wavelength emitted from the first LED module 20 of the first head 15 may be diffused by the light guide 60 at the predetermined diffusion angle, and irradiated on the irradiation area with the uniform illuminance.

According to the inventive concept, the light of the specific wavelength may be used to prevent and manage the skin problem and the external auditory meatus disease of the pet, and the phototherapy treatment apparatus for the pet may be conveniently carried and used regardless of the place.

While the inventive concept has been described with reference to exemplary embodiments, it will be apparent to those skilled in the art that various changes and modifications may be made without departing from the spirit and scope of the inventive concept. Therefore, it should be understood that the above embodiments are not limiting, but illustrative.

What is claimed is:

1. An apparatus for phototherapy treatment of a pet, the apparatus comprising:
a grippable main body having a pair of heads for respectively emitting light;
a plurality of first LED modules respectively disposed in the pair of heads, wherein the plurality of first LED modules irradiate light of a specific wavelength toward a skin and an external auditory meatus of the pet, respectively;
at least one second LED module disposed in one of the pair of heads, wherein the at least one second LED module irradiates light of a wavelength different from the wavelength of the light of the plurality of first LED modules toward the external auditory meatus of the pet; and
a controller disposed in the main body and configured to control the first LED module and the second LED module to respectively irradiate the light.

2. The apparatus of claim 1, wherein the first LED module emits light having a wavelength of 630 nm, and the second LED module emits light having a wavelength of 405 nm.

3. The apparatus of claim 1, wherein the first LED module and the second LED module emit light of different colors, respectively.

4. The apparatus of claim 1, further comprising:
an illumination lamp disposed in at least one of the pair of heads to illuminate the skin or the external auditory meatus of the pet.

5. The apparatus of claim 1, further comprising:
a light guide detachably coupled to the head to diffuse the light emitted from the head.

6. The apparatus of claim 5, wherein the light guide has a shape of a hollow cone having a constant gradient and having both sides open.

7. The apparatus of claim 1, further comprising:
a switch disposed on the main body to turn on or off the first LED module and the second LED module.

8. The apparatus of claim 1, further comprising:
a timer for measuring a time,
wherein the controller is configured to control the first LED module and the second LED module irradiating the light to be turned off when a time set by the timer elapses.

9. The apparatus of claim 1, further comprising:
a battery disposed in the main body to supply power to the first LED module, the second LED module, and the controller.

10. The apparatus of claim 9, further comprising:
a charging terminal disposed in the main body to charge the battery.
